(19) United States
(12) Patent Application Publication
(10) Pub. No.: US 2023/0375547 A1
(43) Pub. Date: Nov. 23, 2023

(54) METHODS, DEVICES, AND RELATED ASPECTS FOR DETECTING EBOLA VIRUS

(71) Applicant: ARIZONA BOARD OF REGENTS ON BEHALF OF ARIZONA STATE UNIVERSITY, Scottsdale, AZ (US)

(72) Inventors: Xiahui CHEN, Mesa, AZ (US); Shoukai KANG, Seattle, WA (US); MD Ashif IKBAL, Tempe, AZ (US); Zhi ZHAO, Mesa, AZ (US); Liangcai GU, Seattle, WA (US); Chao WANG, Chandler, AZ (US)

(73) Assignee: ARIZONA BOARD OF REGENTS ON BEHALF OF ARIZONA STATE UNIVERSITY, Scottsdale, AZ (US)

(21) Appl. No.: 18/247,161

(22) PCT Filed: Sep. 29, 2021
(86) PCT No.: PCT/US2021/052692

§ 371 Date: Mar. 29, 2023

Related U.S. Application Data
(60) Provisional application No. 63/085,004, filed on Sep. 29, 2020.

(51) Int. Cl. G01N 33/569; G01N 33/543

(52) U.S. Cl. CPC G01N 33/56983; G01N 33/54346; G01N 33/54386; G01N 2333/08; G01N 2469/10

(57) ABSTRACT

Provided herein are methods of detecting Ebola virus in a sample. The methods include contacting the sample with a plurality of gold nanoparticles (AuNPs) that are conjugated with antibodies, or antigen binding portions thereof, that bind to an epitope of a secreted glycoprotein (sGP) from the Ebola virus under conditions sufficient for the antibodies, or the antigen binding portions thereof, to bind to the epitope of the sGP from the Ebola virus in the sample to produce bound sGP. The methods also include detecting the sGP from the Ebola virus when aggregations of the bound sGP form with one another. Related reaction mixtures, devices, kits, and systems are also provided.

### CROSS-REFERENCE TO RELATED APPLICATIONS

This application claims priority to U.S. Provisional Patent Application Ser. No. 63/085,004, filed Sep. 29, 2020, the disclosure of which is incorporated herein by reference.

### STATEMENT REGARDING FEDERALLY SPONSORED RESEARCH OR DEVELOPMENT

This invention was made with government support under 1809997, 1847324, 2020464, and 1542160 awarded by the National Science Foundation and R35 GM128918 awarded by the National Institutes of Health. The government has certain rights in the invention.

### BACKGROUND

Zaire ebolavirus (EBOV), a negative RNA-strand virus classified into Filoviridae family with four other known etiological subspecies, is the pathogen that causes hemorrhagic Ebola virus disease (EVD) with statistical fatality rate ranging from 45% to 90%. Zaire ebolavirus accounted for the first EVD epidemic in 1976 and the most massive 2014-2016 outbreak in West Africa, which caused 28,646 infected cases and 11,323 deaths. In a recent massive epidemic as of 6 Aug. 2019, another 2,781 cases and 1,866 deaths have been reported in Democratic Republic of the Congo. On 1 Jun. 2020, the World Health Organization (WHO) declared the 11^{th} outbreak ongoing in Democratic Republic of the Congo. Accumulated knowledge of EBOV outbreaks in recent decades indicates that the outbreak mode is sporadic and fast spreading, accompanied with fast virus genome evolution and high local fatality rate in underdeveloped regions. Although potential antivirus medicine, therapeutic antibodies and vaccines have been reported, there is no approved antiviral drugs by US Food and Drug Administration (FDA) and supportive care remains the only option for most treatments. These combined facts highlight the stringent diagnostic requirements in fast screening and accurate detection of EBOV for current and future epidemic preparedness and rapid, effective containment of EBOV outbreak.

Currently, most commercially available and FDA or WHO approved EVD diagnostics are based on reverse-transcription polymerase chain reaction (RT-PCR) assay, enzyme-linked immunosorbent assay (ELISA) and colloidal gold or fluorescence based rapid immunoassay. RT-PCR and ELISA generally require skilled medical practitioners operating bulky equipment and following elaborate diagnostics protocols that can takes up to 5 hours to complete. In addition, centralized laboratories with well-established EVD diagnostics capacities are generally greatly limited due to the strict biosafety regulations (BSL-4), which significantly reduce the diagnostics throughput and increase diagnostic cost taking into account the biological sample shipment charges from the epidemic region to the laboratories. These methods, although widely used in clinical diagnostics, generally lack the stringent requirements of quick, user-friendly, electricity-free and low cost in field point-of-care (POC) solutions to EVD diagnostics in medical resource limited regions.

Rapid lateral flow immunochromatographic technology has been widely adopted in POC applications. It generally meets POC requirements such as fast screening speed, low cost and zero electricity consumption. However, its detection sensitivity is relatively low (up to hundreds of ng/ml in antigen protein detection) compared to RT-PCR and ELISA and entails qualitative analysis unless coupled to readout devices. In addition, there have been confirmed false positive cases (as high as 20%) from field validation, which can leave patients exposed to nosocomial EVD transmission, potentially limiting its application in field-tests.

Researchers have reported improved sensitivity using Fe₃O₄ magnetic nanoparticles and multifunctional nanospheres in certain applications. However, these approaches typically utilize complicated detecting agent preparations and relatively high associated diagnostic costs. Besides these well adopted clinical diagnostics methods, several bioassays have been reported in current literature, with most of the works focusing on antigen-antibody conjugation or oligonucleotide hybridization and sensing signal transduced through florescence resonance energy transfer, single-particle interferometric reflectance imaging, opto-fluidic nanoplasmonic biosensors, nanoantenna array, memristor and field-effect transistors. Some of these methods demonstrate very sensitive detection (limit of detection reaching femtomolar) attributed to delicate signal transduction mechanism. On the other hand, such high sensitivity is often at the cost of elaborate sample preparation and complicated characterization, creating challenges for low cost miniaturized POC applications.

Accordingly, there is a need for additional methods, and related aspects, of detecting Ebola virus that are low cost, sensitive, easy-to-use, and which yield rapid POC results, particularly under low resource conditions.

### SUMMARY

The present disclosure relates, in certain aspects, to ultra-sensitive gold nanoparticle (AuNP) and/or other plasmonic metal nanoparticle (MNP) colorimetric assays for portable and early-stage EBOV detection. In some embodiments, the AuNP and/or other MNP surfaces are functionalized with high affinity antibodies to EBOV related biomarkers. In these embodiments, the biomarkers generally induce the crosslinking of MNP leading to large MNP aggregate formation and precipitation, resulting in a decrease of MNP monomer concentration in solution and accordingly, a visible change in solution transparency.

In some embodiments, secreted glycoprotein (sGP) is used as a biomarker to be detected. The sGP is a disulfide linked homodimer expressed from the EBOV GP gene, taking approximately 80% of the GP gene transcription in a typical EBOV. Functioning in antigenic decoy activity during infection, sGPs are secreted in significant amounts inside host cells and released into extracellular medium, which is among the first virion related protein readily detected in patient's blood (typically at μg/ml levels). By combining the AuNP and/or other MNP solution-based assay with EVD early-stage infection biomarker sGP, some embodiments of the present disclosure provide a low cost, ultra-sensitive, colorimetric and easy-to-use EVD diagnostic assay that can detect sGP concentration down to, for example, about 1.1 μg/ml (10 nM) by naked eye or visual inspection and about 5.8 ng/ml (52.7 pM) by portable UV-visible spectrometer detection, which can be readily applied in EVD point-of-care diagnostics, especially in underdeveloped regions. These and other aspects will be apparent upon a complete review of the present disclosure, including the accompanying figures.

In one aspect, the present disclosure provides a method of detecting a virus in a sample. The method includes contacting the sample with a plurality of gold nanoparticles (AuNPs) and/or other plasmonic metal nanoparticles (MNPs) that are conjugated with one or more antibodies, or antigen binding portions thereof, that bind to an epitope of a viral antigen of or from the virus under conditions sufficient for the one or more antibodies, or the antigen binding portions thereof, to bind to the epitope of the viral antigen of or from the virus in the sample to produce bound viral antigen. The method also includes detecting the viral antigen of or from the virus when one or more aggregations of the bound viral antigen form with one another, thereby detecting the virus in the sample. Related reaction mixtures, devices, kits, and systems are also provided.

In another aspect, the present disclosure provides a method of detecting Ebola virus in a sample. The method includes contacting the sample with a plurality of gold nanoparticles (AuNPs) and/or other MNPs that are conjugated with one or more antibodies (e.g., monoclonal antibodies or the like), or antigen binding portions thereof, that bind to an epitope of a secreted glycoprotein (sGP) from the Ebola virus under conditions sufficient for the one or more antibodies, or the antigen binding portions thereof, to bind to the epitope of the sGP from the Ebola virus in the sample to produce bound sGP. In some embodiments, a given antibody, or antigen binding portion thereof, comprises a mass of between about 10 kDa and about 20 kDa (e.g., about 11 kDa, about 12 kDa, about 13 kDa, about 14 kDa, about 15 kDa, about 16 kDa, about 17 kDa, about 18 kDa, or about 19 kDa). In some embodiments, a given antibody comprises an equilibrium dissociation constant (K_{D}) on the order of between about 1 nM and about 100 nM (e.g., about 5 nM, about 10 nM, about 15 nM, about 20 nM, about 30 nM, about 40 nM, about 50 nM, about 60 nM, about 70 nM, about 80 nM, or about 90 nM). In some embodiments, the plurality of AuNPs and/or other MNPs is conjugated with one or more nanobodies. In some embodiments, the sGP is sGP49. The method also includes detecting the sGP from the Ebola virus when one or more aggregations of the bound sGP form with one another, thereby detecting the Ebola virus in the sample.

In some embodiments, the detection step comprises determining a change in absorbance at a resonance wavelength of the AuNPs and/or the other MNPs. In some embodiments, the method includes quantifying an amount of the sGP and/or the Ebola virus in the sample. In some embodiments, the method further includes centrifuging the aggregations of the bound sGP prior to and/or during the detecting step. In some embodiments, the method further includes freezing the aggregations of the bound sGP prior to the detecting step. In some embodiments, the method includes drop casting the aggregations of the bound sGP prior to the detecting step. In some embodiments, the method includes obtaining the sample from a subject. In some embodiments, the method includes administering one or more therapies to the subject when the Ebola virus is detected in the sample. In some embodiments, the method includes detecting the Ebola virus within about 20 minutes or less of obtaining the sample from the subject. In some embodiments, the method includes repeating the method using one or more longitudinal samples obtained from the subject (e.g., to monitor a course of therapy and/or progression of the Ebola virus disease over time).

Essentially any sample type is used in performing the methods disclosed herein. In some embodiments, for example, the sample comprises blood, plasma, or serum. In some embodiments, the sample comprises saliva or sputum. In some embodiments, the sample comprises urine.

In some embodiments, the detecting step comprises measuring a colorimetric change when the one or more aggregations of the bound sGP form with one another. In some embodiments, the method includes visually detecting the colorimetric change when the one or more aggregations of the bound sGP form with one another. In some embodiments, the method includes detecting the colorimetric change when the one or more aggregations of the bound sGP form with one another using a spectrometer. In some embodiments, a concentration of sGP in the sample is from about 1 μM to about 100 fM. In some embodiments, a concentration of sGP in the sample is about 100 pM or less. In some embodiments, the AuNPs and/or the other MNPs comprise a substantially spherical shape. In some embodiments, the AuNPs and/or the other MNPs comprise a cross-sectional dimension of between about 10 nm and about 1000 nm.

In another aspect, the present disclosure provides a reaction mixture that includes a sample comprising Ebola virus, and a plurality of gold nanoparticles (AuNPs) and/or other plasmonic metal nanoparticles (MNPs) that are conjugated with one or more antibodies, or antigen binding portions thereof, that bind to an epitope of a secreted glycoprotein (sGP) from the Ebola virus in the sample.

In another aspect, the present disclosure provides a device that includes at least one reaction chamber (e.g., a body structure comprising an array of wells or the like) or substrate comprising a plurality of gold nanoparticles (AuNPs) and/or other plasmonic metal nanoparticles (MNPs) that are conjugated with one or more antibodies, or antigen binding portions thereof, that bind to an epitope of a secreted glycoprotein (sGP) from an Ebola virus when the reaction chamber or substrate receives a sample that comprises the Ebola virus under conditions sufficient for the one or more antibodies, or the antigen binding portions thereof, to bind to the epitope of the sGP from the Ebola virus in the sample to produce bound sGP and one or more aggregations of the bound sGP to produce a colorimetric change in the reaction chamber. In some embodiments, the one or more aggregations of the bound sGP are drop cast in or on the reaction chamber or substrate. In some embodiments, a kit includes the device.

In another aspect, the present disclosure provides a system that includes a device, comprising at least one reaction chamber or substrate comprising a plurality of gold nanoparticles (AuNPs) and/or other plasmonic metal nanoparticles (MNPs) that are conjugated with one or more antibodies, or antigen binding portions thereof, that bind to an epitope of a secreted glycoprotein (sGP) from an Ebola virus when the reaction chamber receives a sample that comprises the Ebola virus under conditions sufficient for the one or more antibodies, or the antigen binding portions thereof, to bind to the epitope of the sGP from the Ebola virus in the sample to produce bound sGP. The system also includes an electromagnetic radiation detection apparatus positioned, or positionable, within sufficient proximity to the device to detect one or more colorimetric changes produced in or on the reaction chamber or substrate when one or more aggregations of the bound sGP form with one another in or on the reaction chamber or substrate. In some embodiments, the electromagnetic radiation detection apparatus comprises a spectrometer. In some embodiments, the electromagnetic radiation detection apparatus comprises a microscope. In some embodiments, the electromagnetic radiation detection apparatus comprises a light-emitting diode (LED) that transmits light into and/or through the reaction chamber or substrate and a photodetector that detects light from the reaction chamber or substrate. In some embodiments, the system includes a device holder that is structured to hold the device, which device holder comprises at least one optical channel through which light is transmitted.

### BRIEF DESCRIPTION OF THE DRAWINGS

The accompanying drawings, which are incorporated in and constitute a part of this specification, illustrate certain embodiments, and together with the written description, serve to explain certain principles of the methods, reaction mixtures, devices, kits, and related systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of detecting Ebola virus according to some aspects disclosed herein.

FIG. 2 (panels A-F) show aspects of a sensing mechanism of an AuNP solution-based assay according to some aspects disclosed herein. (a) Schematic showing sGP mediated AuNP aggregation. (b) Schematic showing sGP49 surface functioned AuNP aggregation and precipitation induced supernatant color change. (c) Visual image of 60 nm AuNP solution-based colorimetric assay in detecting 100 nM and 10 nM sGP in 1×PBS. (d) Visual image of 60 nm AuNP solution-based colorimetric assay drop casted on a 1 mm thick glass slide in detecting sGP concentration from 100 fM to 1 μM in logarithmic scale. (e) Cryo-TEM image of 80 nm sGP49 surface functioned AuNP solution in the absence of sGP. (f) Cryo-TEM image of a typical aggregate in the precipitates of 80 nm AuNP solution mixing with 1 nM sGP in 1×PBS.

FIG. 3 (panels A-F) show aspects of the detection of sGP in 1×PBS using 80 nm AuNP solution-based colorimetric assay according to some aspects disclosed herein. (a) A lab-based UV-visible spectrometer system consisting of Horiba iHR320 spectrometer (left side of image) and Olympus BX53 microscope (right side of image). (b) Schematic of the characterization of AuNP solution-based assay using PDMS template as sample cuvette. (c) Visual image taken by smartphone of 80 nm AuNP solution samples loaded on PDMS template, 3 hours after AuNP mixing with 1 pM to 1 μM sGP in 1×PBS. Concentrations were assigned in logarithmic scale. (d) Absorbance spectra of samples shown in (c), measured by our UV-visible spectrometer system shown in (a). (e) Absorbance maximum plot for AuNP assay in detecting sGP and GP1,2 with concentration from 1 pM to 1 μM. Absorbance maximum is derived from spectra at AuNP resonance wavelength 559 nm. Reference sample (no sGP or GP1,2) is shown as 10-4 nM for comparison. (f) Time-resolved absorbance measured at 559 nm of 80 nm AuNP assay in detecting 10 nM sGP in 1×PBS. Detection time is defined as the time counted from the moment AuNP solution is mixed with sGP analyte.

FIG. 4 (panels A-E) show aspects of the UV-visible spectrometer, SEM and dark field scattering characterization of drop cast samples according to some embodiments disclosed herein. (a) Absorbance spectra of 80 nm AuNP solution-based colorimetric assay solutions (sGP concentration 1 pM to 1 μM in logarithmic scale) drop casted on a 1 mm thick glass slide, measured by Horiba iHR320 spectrometer. (b) Absorbance maximum derived from spectra in (a) at AuNP resonance wavelength 573 nm. Reference sample (no sGP) is shown as 10⁻⁴ nM for comparison. Inset figure shows the visual image of assay solutions drop casted glass slide. (c) Number count of bright spots from particle scattering in dark field scattering images taken for drop cast samples on gold film. sGP concentration is from 1 pM to 1 μM in logarithmic scale. Inset figure shows visual image of 80 nm AuNP assay solutions drop casted on 100 nm gold film deposited by thermal evaporator on silicon substrate. (d) SEM image of drop cast samples on gold film, taken by Hitachi S-4700 field emission SEM at 5 kV accelerating voltage. (e) 80 nm AuNP density derived from SEM images taken for drop cast samples on gold film.

FIG. 5 (panels A-D) show aspects of a centrifuge enhanced fast detection of sGP in 1×PBS using 80 nm AuNP solution-based colorimetric assay according to some embodiments disclosed herein. (a) Schematic showing fast AuNP crosslinking mediated by centrifugal concentration. (b) Absorbance spectra of AuNP assay in detecting sGP in 1×PBS with concentration from 1 pm to 1 μM in logarithmic scale, following centrifuge enhanced fast detection method with 20 minutes crosslinking time. (c) Absorbance maximum plot for AuNP assay in detecting sGP with concentration from 1 pM to 1 μM. Absorbance maximum is derived from spectra in (b) at AuNP resonance wavelength 559 nm. Reference sample is shown as 10⁻⁴ nM for comparison. (d) Influence of crosslinking time (detection time) on absorbance of 80 nm AuNP assay (measured at 559 nm) in detecting 10 nM sGP in 1×PBS. Crosslinking time is defined as time between centrifugal concentration and vortex resuspension of AuNP. Absorbance of reference sample is plotted for comparison. Inset shows narrowed absorbance range for better contrast.

FIG. 6 (panels A-D) show aspects of a point-of-care demonstration of detecting sGP in PBS buffer diluted fetal bovine serum (1:5) using miniaturized portable UV-visible spectrometer measurement system according to some embodiments disclosed herein. (a) POC-based UV-visible spectrometer system consisting of a laptop (right side of image), lamp source module (left side of image), Ocean Optics Flame spectrometer (right front) and alignment clamps (middle of image). The PDMS template loaded sample is placed in the gap between Oceanview signal reading waveguide (right) and light source head (left). (b) Absorbance spectra of PDMS template loaded samples measured by Ocean Optics spectrometer. sGP concentration is from 1 pM to 1 μM in logarithmic scale. (c) Absorbance spectra of samples measured by Horiba iHR320 spectrometer. (d) Absorbance maximum derived from spectra in (b) and (c) at AuNP resonance wavelength 559 nm. sGP concentration is scanned from 1 pM to 1 μM in logarithmic scale. Reference sample (no sGP) is shown as 10⁻⁴ nM for comparison.

FIG. 7A shows a schematic (left) and an actual photo image (right) of an LED-photodetector measurement system, which includes a LED circuit, a photodetector circuit and a 3D printed Eppendorf tube holder according to one exemplary embodiment. The LED and photodetector are embedded into two opposite recesses of an Eppendorf tube holder respectively. A channel is fabricated inside the Eppendorf tube holder to form optical path from the LED to the photodetector for signal measurement.

FIG. 7B are plots of electronic signals measured for assays detecting sGP in PBS buffer diluted fetal bovine serum (5-fold) using the LED-photodetector measurement system from FIG. 7A, shown in black colored points. Olympus BX53 measured absorbances for the same assays are plotted in blue color for reference.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a.” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, reaction mixtures, devices, kits, and systems, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a secreted glycoprotein (sGP) of Ebola virus, e.g., a molecule in which one or more immunoglobulin chains is not full length, but which specifically binds to sGP. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Conjugate: As used herein, “conjugate” refers to a reversible or irreversible connection between two or more substances or components. In some embodiments, for example, gold nanoparticles (AuNPs) are connected to antibodies and/or to antigen binding portions thereof. In some embodiments, AuNPs are conjugated with antibodies and/or to antigen binding portions thereof via one or more linker compounds.

Detect: As used herein, “detect,” “detecting,” or “detection” refers to an act of determining the existence or presence of one or more target analytes (e.g., a secreted glycoprotein (sGP) from an Ebola virus) and/or a pathogen (e.g., an Ebola virus) in a sample.

Epitope: As used herein, “epitope” refers to the part of an antigen (e.g., a secreted glycoprotein (sGP) from an Ebola virus) to which an antibody and/or an antigen binding portion binds.

Reaction Mixture: As used herein, “reaction mixture” refers a mixture that comprises molecules and/or reagents that can participate in and/or facilitate a given reaction or assay. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction or assay components.

Sample: As used herein, “sample” means anything capable of being analyzed by the methods, devices, and/or systems disclosed herein.

Subject: As used herein, “subject” refers to an animal, such as a mammalian species (e.g., human, dog, cat) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). In certain embodiments, the subject is a human. In certain embodiments, the subject is a companion animal, including, but not limited to, a dog or a cat. A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, “system” in the context of analytical instrumentation refers a group of objects and/or devices that form a network for performing a desired objective.

### DETAILED DESCRIPTION

Ebola virus (EBOV) is a highly virulent pathogen, causing severe hemorrhagic fever in humans with 45-90% lethality. In certain aspects, the present disclosure provides a simple yet sensitive colorimetric assay for portable, fast and low-cost EBOV detection in serum or other sample types. In some embodiments, the assay includes gold nanoparticles (AuNPs) conjugated with a high-affinity antibody (secreted glycoprotein 49 (sGP49), 17.1 kDa, K_{D}=41 nM), which is specific to EBOV secreted glycoprotein (sGP) and capable of bridging AuNPs into aggregates upon sGP binding. This aggregation results in a decrease of monomer AuNPs concentration and accordingly, a visible change in solution color transparency. In some embodiments, for example, 100 pM or less sGP can be detected by simply analyzing the absorbance (e.g., 3 mm optical path) of 80 nm (e.g., resonance at 559 nm) AuNPs, presenting about a 8.8% change in absorbance at the resonance wavelength. Moreover, in some embodiments, the reduction of absorbance at the resonance wavelength shows accurate quantitative relation with sGP concentration in a large detection dynamic range from about 100 nM (11 μg/ml) to about 10 pM (1.1 ng/ml), which overlaps with the sGP concentration level observed clinically in patients' blood.

In addition, in some embodiments, the assays disclosed herein can deliver accurate detection results in about 20 minutes or less by accelerating AuNP crosslinking, for example, using centrifuge concentration. Multiple characterization methods, including scanning electron microscopy (SEM) and dark field scattering imaging, among other techniques, can be applied for quantitative analysis in sGP detection with less than one nM sensitivity and accuracy. In some embodiments, the present disclosure also demonstrates the feasibility of detecting sGP in serum or other sample types with miniaturized portable UV-visible spectrometers for point-of-care EBOV detection. In some embodiments, for example, the AuNP solution-based colorimetric assays and other aspects disclosed herein provide ultra-high sensitivity, low cost and electricity free colorimetric detection, which can be readily utilized for point-of-care detection of Ebola virus in remote pandemic regions. These and other aspects will be apparent upon a complete review of the present disclosure, including the accompanying figures.

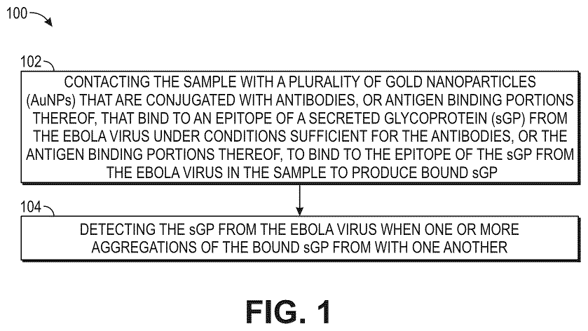

To illustrate, FIG. 1 is a flow chart that schematically shows exemplary method steps of detecting Ebola virus in a sample according to some embodiments. As shown, method 100 includes contacting the sample with a plurality of gold nanoparticles (AuNPs) that are conjugated with one or more antibodies (e.g., monoclonal antibodies or the like), or antigen binding portions thereof, that bind to an epitope of a secreted glycoprotein (sGP) from the Ebola virus under conditions sufficient for the antibodies, or the antigen binding portions thereof, to bind to the epitope of the sGP from the Ebola virus in the sample to produce bound sGP (step 102). Essentially any sample type is used in performing method 100. In some embodiments, for example, the sample comprises blood, plasma, or serum. In some embodiments, the sample comprises saliva or sputum. In some embodiments, the sample comprises urine. In some embodiments, a given antibody comprises a mass of about 17 kDa. In some embodiments, a given antibody comprises an equilibrium dissociation constant (KD) of about 40 nM. The production of antibodies and antigen binding portions thereof suitable for use with methods, devices, and other aspects of the present disclosure are described further herein or otherwise know to a person having ordinary skill in the art.

Method 100 also includes detecting the sGP from the Ebola virus when aggregations of the bound sGP form with one another to thereby detect the Ebola virus in the sample (step 104). In some embodiments, the detection step includes determining a change in absorbance at a resonance wavelength of the AuNPs. In some embodiments, method 100 includes quantifying an amount of the sGP and/or the Ebola virus in the sample. In some embodiments, method 100 further includes centrifuging the aggregations of the bound sGP prior to and/or during the detecting step. In some embodiments, method 100 further includes freezing the aggregations of the bound sGP prior to the detecting step. In some embodiments, method 100 includes drop casting the aggregations of the bound sGP prior to the detecting step. In some embodiments, method 100 includes obtaining the sample from a subject. In some embodiments, method 100 includes administering one or more therapies to the subject when the Ebola virus is detected in the sample. In some embodiments, method 100 includes detecting the Ebola virus within about 20 minutes or less of obtaining the sample from the subject. The method 100 may detect the Ebola virus within 25 minutes, 24 minutes, 23 minutes, 22 minutes, 21 minutes, 20 minutes, 18 minutes, 16 minutes, 14 minutes, 12 minutes, 10 minutes, 8 minutes, 6 minutes, 5 minutes, 4 minutes, 3 minutes, 2 minutes 1 minute, or any range between these values. In some embodiments, method 100 includes repeating the method using one or more longitudinal samples obtained from the subject (e.g., to monitor a course of therapy and/or progression of the Ebola virus disease over time). In some embodiments, the detecting step comprises measuring a colorimetric change when the one or more aggregations of the bound sGP form with one another. In some embodiments, method 100 includes visually detecting the colorimetric change when the one or more aggregations of the bound sGP form with one another. In some embodiments, method 100 includes detecting the colorimetric change when the one or more aggregations of the bound sGP form with one another using a spectrometer. In some embodiments, a concentration of sGP in the sample is about 15 nM or less (e.g., when visually detecting the colorimetric change). In some embodiments, a concentration of sGP in the sample is about 100 pM or less (e.g., when detecting the colorimetric change using a spectrometer). In some embodiments, the AuNPs comprise a substantially spherical shape. In some embodiments, the AuNPs comprise a cross-sectional dimension of between about 10 nm and about 100 nm.

In another aspect, the present disclosure provides a reaction mixture that includes a sample comprising Ebola virus, and a plurality of gold nanoparticles (AuNPs) that are conjugated with one or more antibodies, or antigen binding portions thereof, that bind to an epitope of a secreted glycoprotein (sGP) from the Ebola virus in the sample.

In another aspect, the present disclosure provides a device that includes at least one reaction chamber (e.g., a body structure comprising an array of wells or the like) or substrate comprising a plurality of gold nanoparticles (AuNPs) that are conjugated with one or more antibodies, or antigen binding portions thereof, that bind to an epitope of a secreted glycoprotein (sGP) from an Ebola virus when the reaction chamber or substrate receives a sample that comprises the Ebola virus under conditions sufficient for the one or more antibodies, or the antigen binding portions thereof, to bind to the epitope of the sGP from the Ebola virus in the sample to produce bound sGP and one or more aggregations of the bound sGP to produce a colorimetric change in the reaction chamber. In some embodiments, the one or more aggregations of the bound sGP are drop cast in or on the reaction chamber or substrate. In other embodiments, the one or more aggregations of the bound sGP may be deposited in or on the reaction chamber or substrate in a variety of deposition techniques. The deposition technique may be spin coating, dip coating, spray coating or any other similar technique known to one of skill in the art. In some embodiments, a kit includes the device.

In another aspect, the present disclosure provides a system that includes a device, comprising at least one reaction chamber or substrate comprising a plurality of gold nanoparticles (AuNPs) that are conjugated with one or more antibodies, or antigen binding portions thereof, that bind to an epitope of a secreted glycoprotein (sGP) from an Ebola virus when the reaction chamber receives a sample that comprises the Ebola virus under conditions sufficient for the one or more antibodies, or the antigen binding portions thereof, to bind to the epitope of the sGP from the Ebola virus in the sample to produce bound sGP. The system also includes an electromagnetic radiation detection apparatus positioned, or positionable, within sufficient proximity to the device to detect one or more colorimetric changes produced in or on the reaction chamber or substrate when one or more aggregations of the bound sGP form with one another in or on the reaction chamber or substrate. In some embodiments, the electromagnetic radiation detection apparatus comprises a spectrometer. In some embodiments, the electromagnetic radiation detection apparatus comprises a microscope. Exemplary devices and systems are described further herein.

### Example

Results and Discussion

Sensing Mechanism

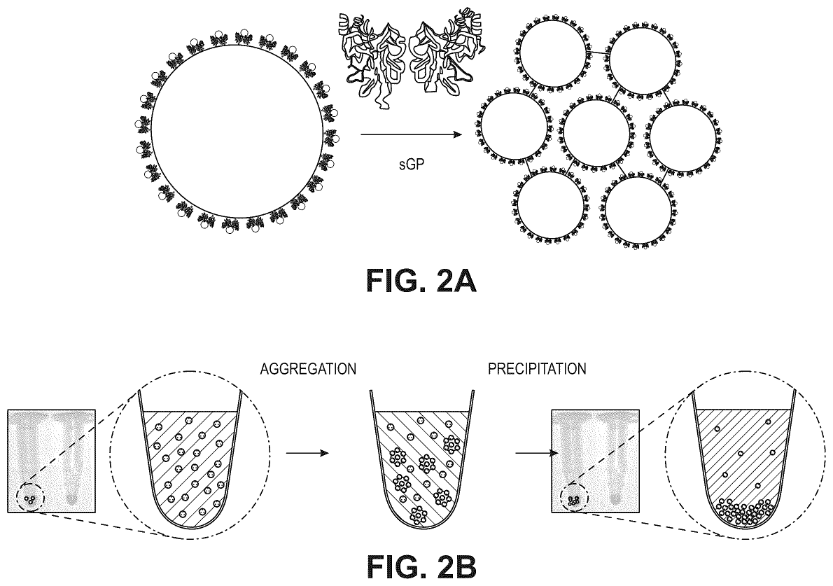

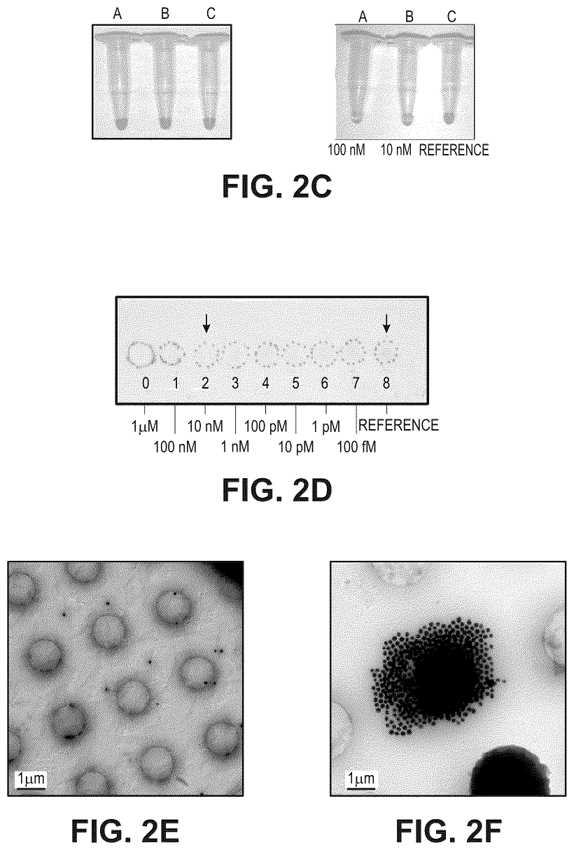

A plasmonic gold nanoparticle based assay was applied to detecting multiple species of analytes from small molecules to large cells through a variety of signal transduction mechanisms, e.g., surface plasmon resonance (SPR), surface enhance Raman spectra (SERS), AuNP enhanced fluorescent, colorimetry, scanning electron microscope (SEM) and so on. Among these mechanisms, analyte mediated crosslinking of AuNP is one of the most straightforward, cost efficient and readily characterizable signal transduction mechanisms. In some embodiments of the present disclosure, the sensing mechanism involves sGP induced crosslinking of highly specific anti-sGP monoclonal antibody sGP49 (17.1 kDa, K_{D}=41 nM) surfaced functionalized AuNP (FIG. 2 (panel a)). The complete schematic of sensing mechanism is shown in FIG. 2 (panel b). Initially, all the AuNP monomers are uniformly dispersed in solution, presenting a reddish color attributed from characteristic LSPR absorbance. Upon mixing with sGP analyte at a certain concentration, AuNP monomers crosslink and form aggregates. Compared to AuNP monomers, the AuNP aggregates do not efficiently scatter light and contribute little to presenting solution color. These AuNP aggregates eventually precipitate at the bottom of the assay container by gravity force. As a result, decreased concentration of AuNP monomers in solution leads to significant color transparency change, indicating that a certain sGP concentration that is detected.

As a colorimetric sensing example, FIG. 2 (panel c) shows the color change for 60 nm AuNP assay solution in detecting 100 nM (11 μg/ml) and 10 nM (1.1 μg/ml) in 1× phosphate-buffered saline (1×PBS), which are typical sGP concentrations in patients' blood. The color contrast between the AuNP assay solution detecting 100 nM and 10 nM and the reference (no sGP mixed) can be easily detected by naked eye or through analysis of images taken by smartphone, leading to portable, electricity-free point-of-care diagnostics. In addition, the assay solution can be drop cast on glass slide (1 mm thick, each drop takes 1 μL) and dried in air for visible examination, as shown in FIG. 2 (panel d). It can be observed that 100 nM and 1 nM sample spot (indexed 1 and 2) has lighter color compared the reference sample spot (no sGP, indexed 8). It is worth noting that the drop cast samples on glass slide gives the same limit of detection by naked eye down to 10 nM compared to solutions in Eppendorf tubes. In addition, drop cast of samples on glass slide shows advantage in prolonged period samples preservation. This example tested the colorimetric sensing performance for drop cast samples over 12 weeks period and the diagnostics results were accurate. This indicates that the AuNP based colorimetric assay is capable of meeting stringent POC diagnostics requirement for sample storage in underdeveloped areas where low temperature sample storage conditions are typically unavailable.

To experimentally confirm the sGP induced AuNP crosslinking and the formation of large sized aggregates at nanoscopic levels, the assay solutions were rapidly frozen and imaged by cryogenic transmission electron microscope (CryoTEM). Cryogenic sample preparation is known to prevent water crystallization and hence preserve protein structures and protein-protein interaction. Under cryogenic conditions, cryoTEM imaging can faithfully reveal the AuNP crosslinking and aggregation network in solutions. Consistent with the sensing mechanism described above, the crosslinking and aggregations of 80 nm AuNP in 10 nM sGP assay sample was confirmed in cryoTEM image FIG. 2 (panel f). The size of the aggregate in the image was approximately 2.45 by 1.92 μm. In the blank control sample, due to the lack of sGP, only 80 nm AuNP monomers were observed (FIG. 2 (panel e)).

sGP Detection

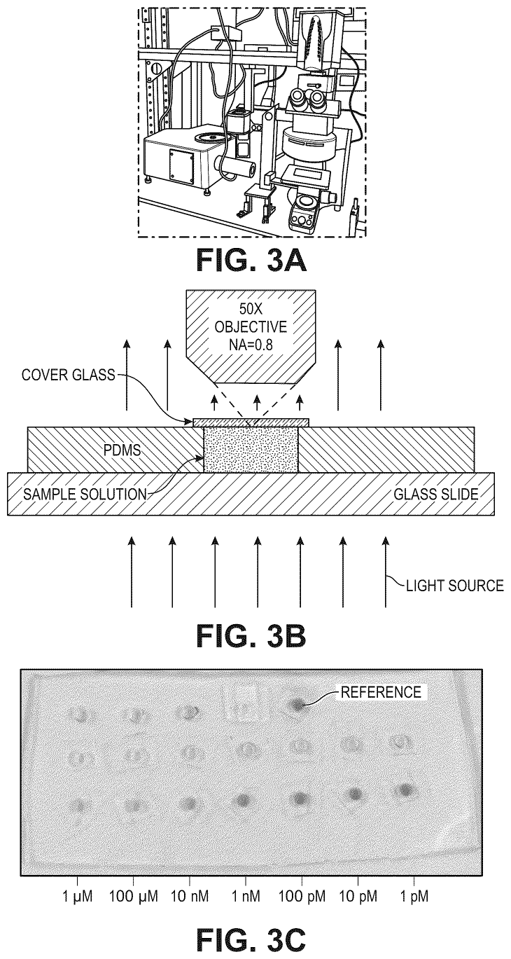

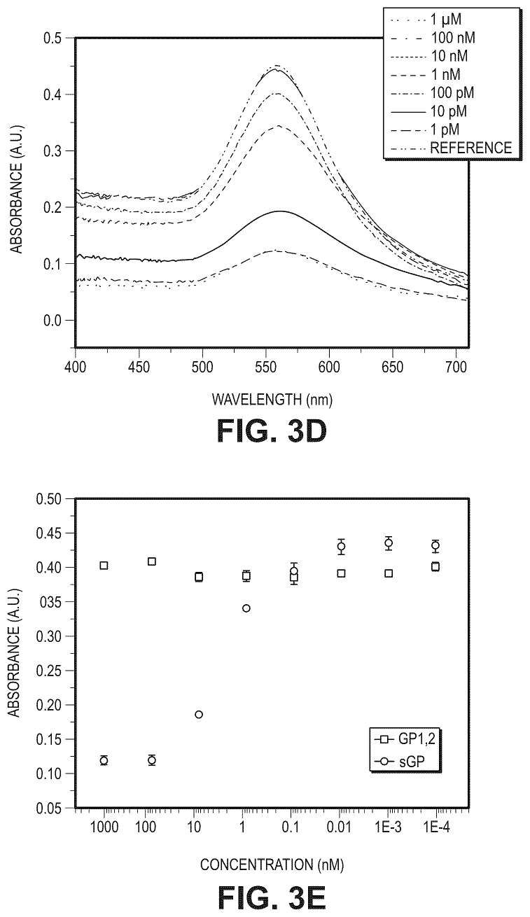

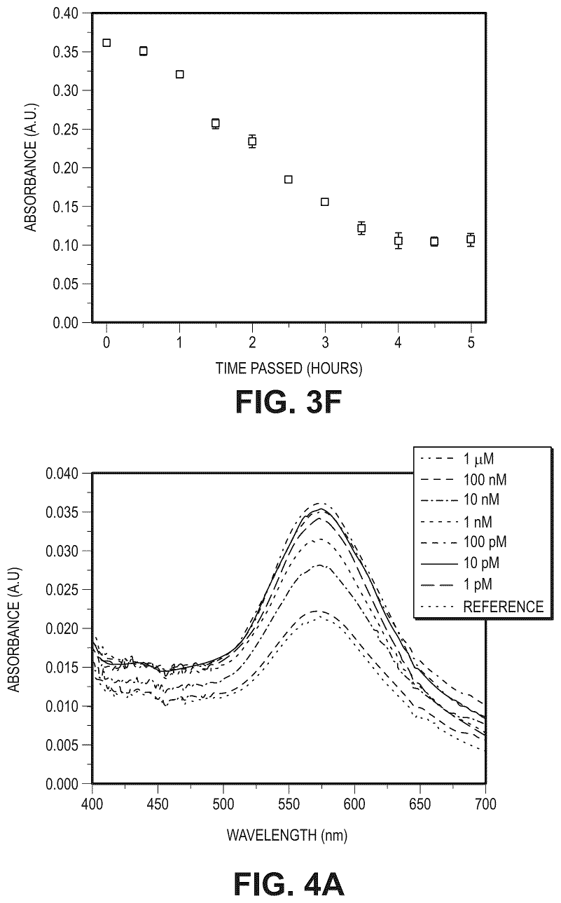

To assess the sGP detection performance of the AuNP solution-based colorimetric assay, a complete quantification of sGP detection using 80 nm diameter AuNP solution (n_{AuNP}=0.036 nM) were characterized by lab based UV-visible spectrometer with hand-made PDMS template as the sample cuvette. Here, the AuNP diameter (40 to 100 nm) was optimized and found that 80 nm AuNP delivered best sensing performance. FIG. 3 (panel a) shows a lab-based UV-visible spectra measurement system consisting of Olympus BX53 microscope for signal collection and Horiba iHR320 UV-visible spectrometer for spectra measurement. As a standard measurement procedure shown in FIG. 3 (panel b), a supernatant sample (5 μL) from an Eppendorf tube contained assay solution was loaded into the 2 mm diameter hole punched through a 3 mm thick PDMS template and sealed by 100 μm thick cover glass. Then, the sample loaded PDMS was placed under 50× objective lens in an Olympus microscope system for UV-visible spectra measurement. FIG. 3 (panel c) shows the image of a PDMS stamp on which 80 nm diameter AuNP solution samples mixed with sGP in 1×PBS (1 pM to 1 μM) were loaded. Visual inspection of the sample template by naked eye indicated that a gradual transparency change of solution redness color started from 1 nM to 1 μM. Significant color contrast of the assay in detecting 10 nM and higher sGP concentration to the reference sample was observed, indicating that clinically representative sGP concentrations can be readily detected by naked eyes.

To accurately characterize the optical properties of the assay solutions. UV-visible absorbance spectra of the 80 nm AuNP assay in detecting sGP in 1×PBS were measured and shown in FIG. 3 (panel d). The absorbance was defined as A=log₁₀(I₀/I), where A is the absorbance, I₀ is the intensity of light passing reference cuvette and I is the intensity of light passing sample cuvette. The measured absorbance spectra for all AuNP assay samples featured a characteristic LSPR resonance peak at 559 nm and the maximum absorbance at the peak position gradually decreased as sGP concentration increased. According to Beer-Lambert law A=εcl (A absorbance, ε extinction coefficient, c concentration, I optical path), the decrease of absorbance indicated a decreased 80 nm AuNP monomer concentration in solution, which was consistent with the sensing mechanism. The absorbance maximum was extracted at the LSPR resonance position for each assay sample and the absorbance was plotted—concentration standard curve in FIG. 3 (panel e) (red datapoints). The quantification of sGP sensing shows that absorbance (A_{reference}=0.432) started to decrease at 10 pM (A_{10 pM}=0.434) and dropped quickly from 100 pM (A_{100 pM}=0.392) to 100 nM (A_{100 nM}=0.112). The absorbance stopped further decreases after sGP concentration reached 100 nM, indicating the completion of AuNP aggregation at high sGP concentration greater than 100 nM. The UV-visible spectrometer characterization of the sensors demonstrated that the 80 nm diameter AuNP solution assay achieved broadband detection dynamic range (10 pM to 100 nM) with the most sensitive linear detection region from 100 pM to 10 nM. As previously mentioned. sGP concentration found in EVD patients' blood is typically in the order of 10 nM (ug/ml). Such broadband dynamic range largely overlaps with the broadband sGP concentration in patients' blood attributed from patient-patient and infection stage variation. While early stage infection of EBOV can lead to significantly lower sGP level, which becomes very challenging to detect for ELISA and lateral flow immunoassays, the standard curve measurement showed the AuNP based colorimetric assay achieved limit of detection (LoD, defined as concentration at A_{reference}−3σ_{A}) down to 45.8 pM (5.0 ng/ml). Such broadband dynamic range and ultrahigh sensitivity indicated that the AuNP solution-based assay is ideal for quantitative sGP detection and feasible to detect early stage EBOV infection.

Specificity of detection is also a significant criterion in disease detection. Low specificity can cause false positive results leading to serious clinical incidents such as nosocomial infection. Here, the specificity of the assay was tested by detecting GP1,2, a homotrimer glycoprotein transcribed from the same GP gene as sGP. The majority of GP1,2 can be found on the virus membrane and a low concentration of GP1,2, also known as shed GP, are found in patients' blood. The close relevance of GP1,2 to sGP made it a good candidate to assess the assay's specificity. As expected, the assay showed very good specificity and no significant absorbance change at any GP1,2 concentration, indicating that no AuNP aggregates formed in the presence of GP1,2 (FIG. 3 (panel e), black datapoint set).

Next, detection speed of the sensing assay was investigated. Absorbance of 80 nm AuNP assay sensing 10 nM sGP in 1×PBS was monitored starting right after mixing sGP analyte and AuNP solution to 5 hours (FIG. 3 (panel f)). The absorbance started to drop after 0.5 hour, indicating the aggregate formation happened within the half hour of mixing and precipitations started to lead the absorbance change after 1 hour. The steady aggregate formation and precipitations led to a nearly linear absorbance drop at a rate of 0.08 hr⁻¹ and the absorbance became stable after 4 hours. The dynamic absorbance test for 10 nM sGP detection indicated that the time for high contrast colorimetric detection is 2 to 3 hours. The detection speed for 40, 60 and 100 nm AuNP assay in detecting 10 nM sGP was also tested and it was found that the absorbance drop rate trend increased for bigger AuNP size, likely due to increased aggregate mass and precipitation speed.

Versatile Characterization Options for AuNP Solution-Based Assay

The assay's broad adaptability to different characterization methods not only improve its diagnostics application feasibility, diagnostics robustness and throughput but also increase the knowledge of the assay's working mechanism. In addition to UV-visible spectrometer characterization of the AuNP assay solution, the assay can be drop cast in solid form and detection can be quantified by a broad spectrum of characterization approaches such as scanning electron microscope imaging, UV-visible spectroscopy and dark field scattering imaging.

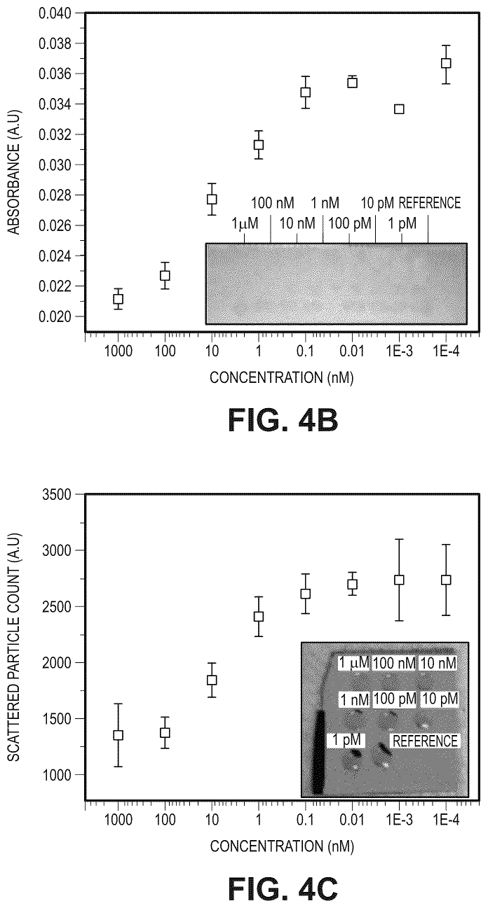

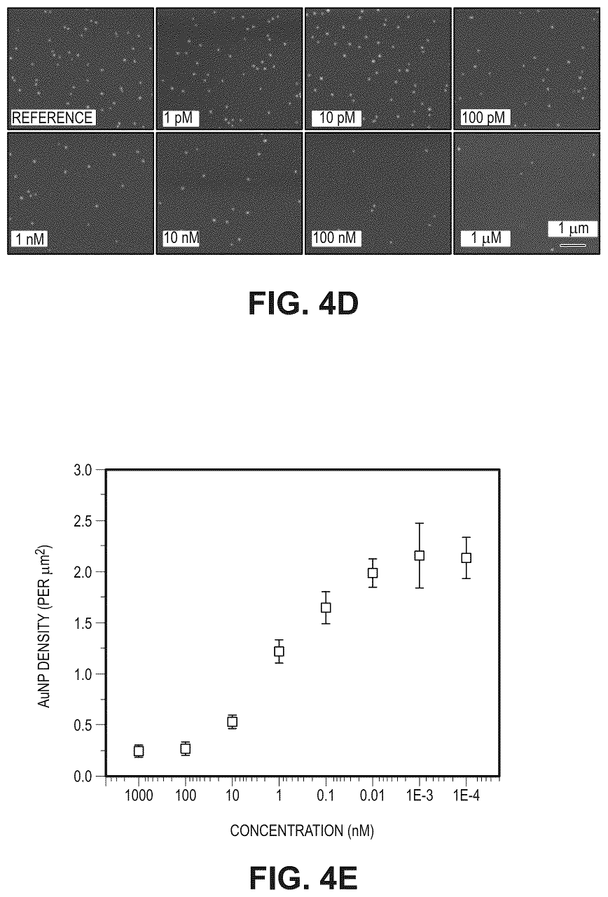

As mentioned above, the assay solution that drop cast and dried can be preserved in the absence of low temperature storage equipment such as fridge for prolonged period. Drop cast can also prevent samples from perishing in shipment from sites of sample collection to the lab-based characterization facilities. Here, the 80 nm AuNP assay solutions (n_{AuNP}=0.036 nM) detecting sGP (1 pM to 1 μM) in 1×PBS were drop cast on a 1 mm thick glass slide and their UV-visible absorbance spectra were measured (FIG. 4 (panel a)). An exemplary image of dried drop cast samples is shown in the inset of FIG. 4 (panel b). The volume for each drop cast sample was 1 μL. A visual inspection by naked eye indicated the transparency of red drop cast spots started to increase at 10 nM and became readily differentiable by naked eye at 100 nM and 1 μM to reference sample. The absorbance spectra for drop cast samples featured AuNP LSPR peaks, similar to those measured from the PDMS template loaded solution samples shown in FIG. 3 (panel d). The extracted absorbance maximum standard curve for drop cast sample is plotted in FIG. 4 (panel b). Due to significantly shorter optical path (estimated ˜300 μm), the absorbance of drop cast samples is one order of magnitude smaller than that of solution samples. Similar to solution samples, the absorbance of the drop cast sample started to decrease at 10 pM (A_{10 pM}=0.035) and gradually saturated at 100 nM (A_{100 nM}=0.023) and 1 μM (A_{1 μM}=0.021). UV-visible spectra characterization of drop cast assay sample showed its effective quantitative detection of sGP down to 350 pM LoD with broad detection dynamic range (100 pM to 1 μM), offering promising solutions to simplification of sample preparation and preservation favored in POC EVD diagnostics.

In recent years, miniaturization of high-end scanning electron microscopy has made significant progress such that direct SEM image-based diagnostics of disease has become feasible. In light of recent SEM image-based diagnostics works, the 80 nm AuNP assay solutions (n_{AuNP}=0.036 nM) detecting sGP (1 pM to 1 μM) in 1×PBS was drop cast on an oxygen plasma treated gold surface and imaged by SEM. The visual image of drop cast samples on gold surface is shown in inset of FIG. 4 (panel c). Each AuNP solution sample drop cast took 1 μL of supernatant solution in an Eppendorf tube. A gold surface was selected due to its high electron conductivity that dramatically improved the contrast and resolution of electron microscopic image. From the SEM image shown in FIG. 4 (panel d), the 80 nm AuNPs were clearly recognizable for each drop cast sample. It can be observed that the density of 80 nm AuNPs gradually drops as the sGP concentration increases. It is worth noting that all AuNPs were dispersed as monomers in all drop cast samples and no large aggregates were observed since the drop cast samples were pipetted from the supernatant of assay solutions, which shows consistency to the sensing mechanisms described herein. The density of AuNP monomers was statistically measured from ten SEM images (total area 10×8.446×5.913 μm²) taken for each sGP concentration and plotted in FIG. 4 (panel e). The density of AuNPs started to decrease at 10 pM (1.97 μm⁻²) until 100 nM (0.26 μm⁻²) and saturated at 1 μM (0.24 μm⁻²), which was in accordance with UV-visible absorbance spectra measurements of both solution samples and glass slide drop cast samples. The limit of detection from SEM characterization (147 pM) was comparable to drop cast sample absorbance characterization (350 pM) but higher than assay solution absorbance characterization (45.8 pM), possibly due to increased variance in nanoscale level characterization and statistical analysis of limited sampling data.

In addition to SEM imaging, the drop cast samples on gold surfaces were characterized by dark field scattering imaging, which has been widely applied in biomolecule sensing and environmental toxin detection. LSPR mediated scattering of incident light from AuNPs on gold surface can directly indicate the density of AuNP based on the density of bright spots shown on dark field scattering image. The area of each image is 62.5×62.5 μm². It can be observed that the density of bright spots drops as sGP concentration increases, indicating the decreased concentration of AuNP in solution. The number of bright spots counted (averaged on 10 images) in each concentration is shown in FIG. 4 (panel f). The counted number of reference sample is 2753.0 and starts to drop at 10 pM (count number 2704.1) until 100 nM (counted number 1376.0) and saturates at 1 μM (count number 1348.1). Such quantitative relation between counted number and sGP concentration for dark field scattering measurement is unsurprisingly consistent with SEM image analysis and UV-visible spectra measurement. The dark field scattering measurement demonstrated its feasibility in sGP detection with sensitivity up to sub 1 nM, serving as a readily accessible characterization method for POC EVD diagnostics.

60 nm AuNP drop cast samples were also characterized by UV-visible spectrometer and 100 nm AuNP drop cast samples by UV-visible spectrometer, SEM and dark field scattering imaging. The measurement results in general show comparable sensitivity (sub 1 nM) to 80 nm AuNP.

As demonstrated above, the characterization of the AuNP solution-based assay by UV-visible spectrometer, SEM and dark field scattering imaging provide accurate quantitative sGP detection results with less than a one nM limit of detection and broad detection dynamic range. The AuNP solution-based assay offers flexible characterization options that can meet a variety of characterization requirements in different diagnostics situations.

Centrifuge Assisted Fast sGP Detection

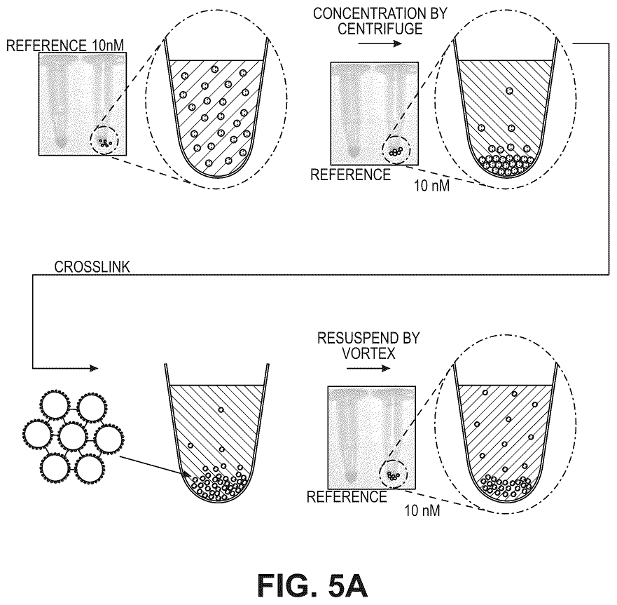

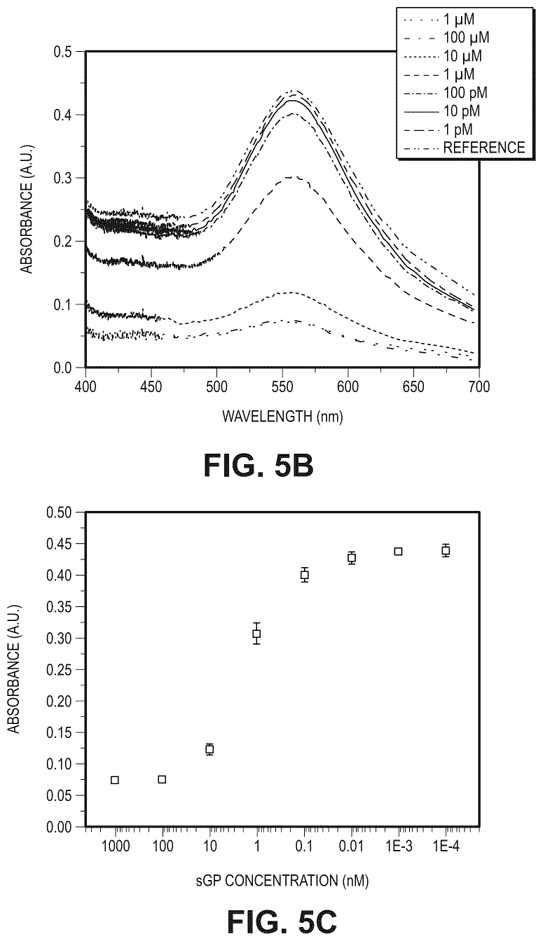

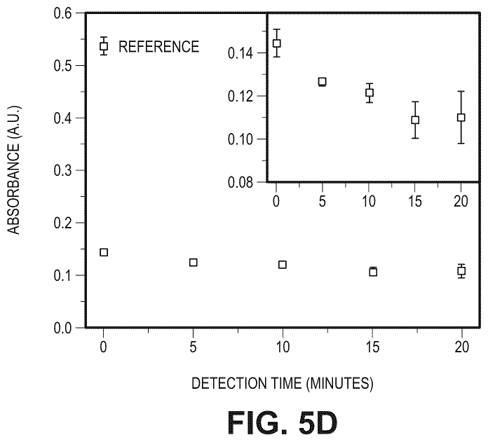

To meet the stringent requirement of fast detection in EVD in field diagnostics, the sensing mechanism was further improved and dramatically reduced the detection time from 3 hours to 20 mins using the same 80 nm AuNP assay. In the sensing mechanism mentioned above, AuNPs are uniformly dispersed in buffer solutions. According to reaction kinetics, the reaction rate is proportional to reactant concentration. Therefore, uniform dispersion of AuNP monomers at low concentration results in slow aggregate formation rate. It is straightforward to dramatically increase the AuNP monomer concentration targeted at higher aggregate formation rate. However, such approach leads to increased assay cost and consumes more analyte to reach comparable signal contrast (saturation absorbance/reference absorbance ratio). In order to simultaneously keep the same assay cost and increase the detection speed, a centrifuge assisted fast detection method is used in some embodiments. The sensing mechanism is shown in FIG. 5 (panel a). After mixing AuNP solution with sGP analyte, a centrifuge step is added to dramatically concentrate AuNP monomers at the bottom of Eppendorf tube. The centrifuge protocol is optimized at 3,500 rpm (1,200×g) for 1 minute to effectively concentrate AuNP monomers while avoiding dense AuNP aggregates formation that prevent sGP diffusion and un-crosslinked AuNP monomer resuspension. AuNP concentration is estimated to increase by at least 3 orders judging from the volume decrease (solution volume to reddish solid precipitates volume at Eppendorf tube bottom). As a result, crosslinking of AuNP at highly concentrated bottom area is greatly accelerated. After 20 minutes of AuNP crosslinking, the assay solution was thoroughly vortexed to resuspend un-crosslinked AuNP monomers back to solution. The visual image taken by smartphone confirmed the increased assay solution transparency as sGP concentration increased starting from 1 nM. The absorbance spectra of AuNP assay in detecting different concentrations of sGP in 1×PBS were measured by UV-visible spectrometer and shown in FIG. 5 (panel b). The absorbance maximum at resonance wavelength 559 nm for each sGP concentration was extracted and plotted in FIG. 5 (panel c). The absorbance-concentration standard curve for the assay undergoing centrifuge concentration was consistent with the standard curve shown in FIG. 5 (panel e), which takes three hours to yield high enough signal contrast (saturation absorbance/reference absorbance ratio: 3.60). The standard curve for centrifuge enhanced assay shows that sGP in 1×PBS starts to cause the absorbance (A_{reference}=0.439) drop at 10 pM (A_{10 pM}+=0.427) and it quickly drops starting from 100 pM (A_{100 pM}=0.400) until 10 nM (A_{10 nM}=0.123) at a rate of 0.139/dec. The absorbance becomes saturated at 100 nM (A_{100 nM}=0.075) and above. Centrifuge assisted fast sGP detection assay not only showed comparable limit of detection (52.8 pM), dynamic ranges (100 nM to 10 pM) and even higher signal contrast (5.92), but tremendously reduced detection time to less than 20 minutes. Moreover, the assay utilizing centrifuge concentration method demonstrated right-after-process detection of 10 nM sGP, i.e., the color contrast is high enough that can be immediately detected by naked eye right after assay is vortexed. FIG. 5 (panel d) shows the UV-visible spectrometer measured absorbance of assay solution in detecting 10 nM sGP in 1×PBS at AuNP resonance position (559 nm) with different detection time. Here detection time was defined as time for AuNP crosslinking between centrifuge and vortex step. The absorbance was 0.145 right after vortex compared to 0.536 of the reference sample. The absorbance gradually decreased to 0.110 as detection time increased to 20 minutes. It is worth noting that 10 nM and higher concentration is typical serum concentration for EVD patients. Further, right-after-process detection can lead to high screening throughput. Therefore, right-after-process detection of 10 nM sGP can be advantageous in fast screening and rapid containment in current and future EVD outbreaks.

In sum, the centrifuge assisted fast detection methods improved the AuNP assay's detection speed without inducing elaborate preparation steps and extra cost, rendering the AuNP assay feasible, for example, in field EVD diagnostics and that can be extensively applied to detection of a variety of analytes based on AuNP crosslinking.

Point-of-Care Detection of sGP in Fetal Bovine Serum

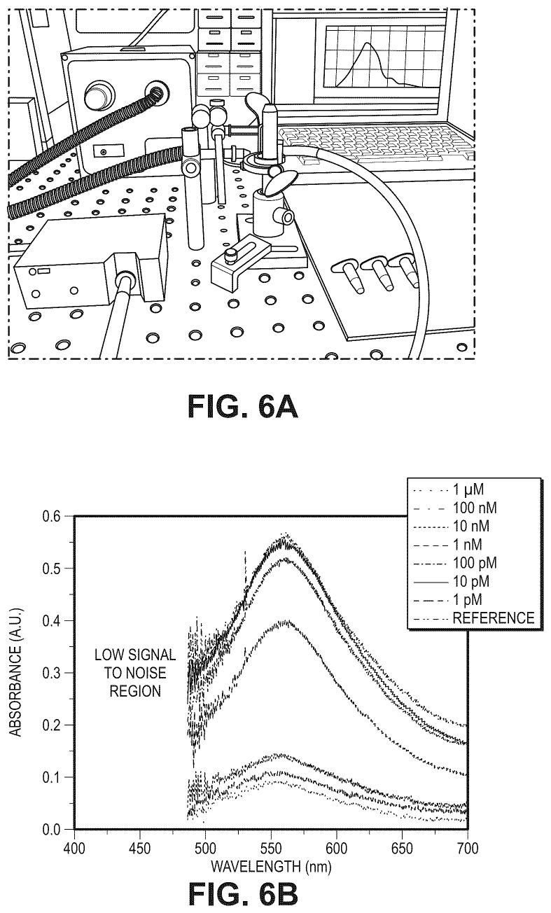

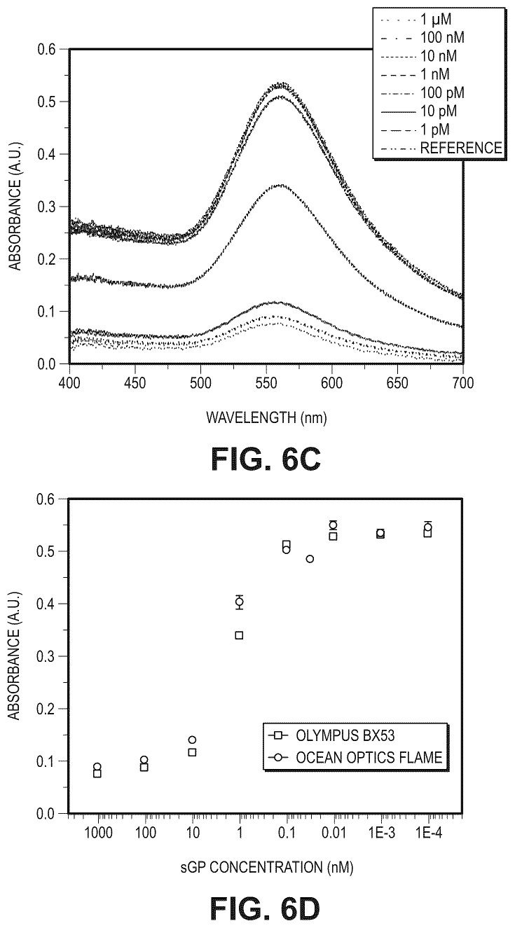

To further meet the goal of POC detection, the feasibility of detecting sGP in a fetal bovine serum sample was demonstrated using miniaturized portable UV-visible spectra measurement system towards stringent in field conditions for EBOV point of care diagnostics. The miniaturized portable UV-visible spectra measurement system consisted of a smartphone sized Ocean Optics UV-visible spectrometer (8.8×6.3×3.1 cm³), a lamp source module (15.8×13.5×13.5 cm³), alignment clamps and a laptop as shown in FIG. 6 (panel a). 80 nm AuNP solutions were mixed with fetal bovine serum containing sGP in 3:1 ratio, thoroughly vortexed and centrifuged at 1,200×g (3,500 rpm) for 1 minutes. After 20 minutes of incubation, the assay solutions were vortexed for 15 seconds and loaded into the 4 mm holes on a 3 mm thick PDMS template. The light from lamp source transmitted through the solution was collected by the waveguide head connecting to the Ocean Optics spectrometer. The absorbance spectra was measured by portable Ocean Optics spectrometer for different sGP concentrations are shown in FIG. 6 (panel b). As a comparison, the same samples were measured by lab-based Olympus BX53 microscope and Horiba iHR320 spectrometer and the absorbance spectra is shown in FIG. 6 (panel c). The absorbance maximums at the resonance wavelength 559 nm are further extracted and plotted in FIG. 6 (panel d). It can be observed that the absorbance maximum measured by Ocean Optics and Horiba spectrometer at given sGP concentration is in general consistent with each other. The difference in absorbance value is within 15.8% and possibly attributed to different collection angles in collecting the transmitted light (10× objective collection versus waveguide collecting from 4 mm aperture). In the case of serum, the absorbance measured from Ocean Optics spectrometer started to decrease at 10 pM (A_{10 pM}+=0.550) and rapidly decreased from 100 pM (A_{100 pM}=0.513) to 10 nM (A_{10 nM}=0.141) at a rate of 0.186/dec. The absorbance did not saturate at higher concentrations (>10 nM) and continued to slowly decrease from 10 nM to 1 μM (A_{1 μM}=0.089) at a rate of 0.026/dec. The 52.7 pM limit of detection was comparable to that of assay detecting sGP in 1×PBS. In conclusion, it was demonstrated that the ultra-sensitive detection of sGP in serum up to 52.7 pM by using our portable UV-visible spectra measurement system. The AuNP based colorimetric assay can be readily applied to POC detection of clinical samples, showing the feasibility of low-cost, easy-to-use and ultra-sensitive quantitative POC diagnostics of EVD in underdeveloped regions.

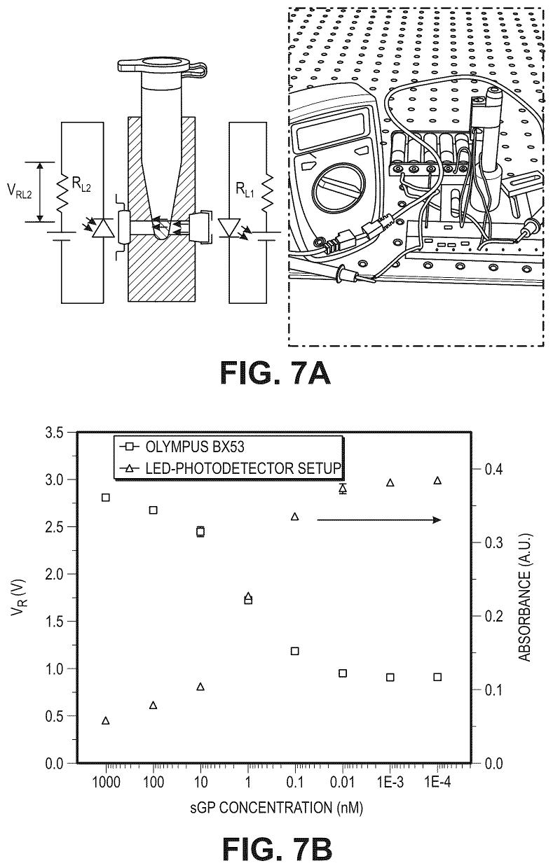

To meet an ultimate POC diagnostics objective, while the measurement system can be miniaturized it is also favorable that the system cost can be as low as possible. Here, we further simplified the measurement system by applying photodetector based electronic signal transduction mechanism, which can dramatically reduce the system cost owning to an economical commercial electronics solution. In this exemplary mechanism, the light source LED emits narrowband wavelength light only at AuNP LSPR wavelength region (λ_{P}=560 nm, FWHM_{P}=40 nm). The light transmits through the assay supernatant, strongly absorbed and scattered and subsequently measured by a photodetector which is only sensitive at a narrowband wavelength centered at AuNP absorbance maximum (λ_{S max}=560 nm, FWHM_{S}=110 nm). Finally, the light intensity is converted to photocurrent or the voltage on a serially connected load resistor that can be measured by a portable multimeter. FIG. 7A (left) shows the schematic and actual image (right) for our LED-photodetector based electronic readout system. A 3D printed Eppendorf tube holder, as small as an AA battery, firmly fixes the assay tube, LED and photodetector to their positions. A small channel is open inside the 3D printed holder to form the optical channel for signal readout. LED and photodetector are powered by AA alkaline batteries (3V and 4.5V respectively) and voltage is properly biased and divided to working points through serial resistors. Electronic readout (voltage over the photodetector load resistor) is measured by a handheld multimeter. For demonstration, we measured different concentrations of sGP in PBS diluted fetal bovine serum (five-fold) using our AuNP assay in combination with centrifuge assisted rapid detection in 20 minutes, which represents a realistic in field test situation. The electronic readings, which reflect the maximum absorbances at 559 nm for different sGP concentrations, are shown in black points in FIG. 7B. As a comparison, the same samples were characterized by lab-based microscope-coupled spectrometer (Horiba iHR320) and the absorbance maximum is plotted in blue points. It is not surprising to observe that the voltages measured show the same monotonous trend as absorbances measured by UV-visible spectrometer. The voltage level can be unambiguously differentiated from 10 pM to 1 μM, showing a broad dynamic range that is similar to referenced UV-visible spectrometer measurement results noted herein. The demonstrated quantitative relation between electronic readout and sGP concentration indicates that our LED-photodetector based electronic readout system is capable of achieving the same quantitative diagnostics results with comparable precision and accuracy as both lab-based and miniaturized UV-visible spectrometer systems. While attaining the same performance, our electronic readout system has certain advantages over spectroscopic readout systems in some respects. First, for example, the cost of the system is reduced attributable to massively manufacturable commercial components, such as LEDs and photodetectors that cost less than $1 each, as well as low cost batteries, resistors and 3D printing parts. Second, the dimension of integrated system can be scaled down to the size comparable to an 0.5 mL Eppendorf tube given the fact that miniaturized electronic components are readily available. Moreover, the electronic readout system also typically outperforms the bare eye colorimetric readout in accuracy and the results are quantitative and digitizable for data analysis. As demonstrated, we not only achieved high sensitivity and large dynamic range in rapid sGP detection, but also reduced the cost and size of assay characterization system. Our efforts on both assay and measurement system optimizations make our work a readily applicable solution for low-cost, easy-to-use, and ultrasensitive POC diagnostics of EVD in, for example, epidemic regions.

### CONCLUSIONS

In conclusion, an ultrasensitive gold nanoparticle solution based colorimetric assay was demonstrated in detecting secreted glycoprotein, an effective early stage biomarker for Ebola virus infection detection. 10 nM sGP can be reliably detected by differentiated assay color transparency with naked eyes. Precise absorbance measurement using UV-visible spectrometer demonstrated that the assay can reach at least 45.8 pM limit of detection and a broad detection dynamic range of four orders of concentration ranging from 10 pM to 100 nM. It was also demonstrated that a miniaturized spectrometer system is effective in detecting serum samples down to 52.7 pM and that detection results can be accurately delivered in 20 minutes with the centrifuge concentration methods. The fast detection speed, high sensitivity, broad detection dynamic range and low cost of the assay combined with its integration to easy-to-use portable spectrometer characterization and flexible adaption to a variety of characterization methods make the gold nanoparticle solution based colorimetric assay highly effective for in field point-of-care diagnostics of early stage Ebola virus infection in remote pandemic regions.

Methods and Materials

Materials

Phosphate-buffered saline (PBS) was purchase from Fisher Scientific. Bovine serum albumin (BSA) and molecular biology grade glycerol and were purchased from Sigma-Aldrich. Fetal bovine serum (FBS) was purchased from Gibco, Fisher Scientific. FBS was avoided heat inactivation to best mimic the state of serum collected in field. Poly(vinyl alcohol) (PVA, M_{W} 9,000-10,000) was purchased from Sigma-Aldrich. Sylgard 184 silicone elastomer kit was purchased from Dow Chemical. The streptavidin surface functioned gold nanoparticles were purchased from Cytodignostics, dispersed in 20% v/v glycerol and 1 wt % BSA buffer solution. Thiolated carboxyl poly(ethylene glycol) linker was self-assembled on AuNP through thiol-sulfide reaction. Streptavidin was then surface functioned through amine-carboxyl coupling mediated by N-Hydroxysuccinimide/1-Ethyl-3-(3-dimethylaminopropyl)carbodiimide (NHS/EDC) chemistry. The biotinylated sGP 49 nanobody and sGP proteins were received from collaborators in University of Washington and dispersed in 1×PBS buffer. DNase/RNase-free distilled water used in experiments and was purchased from Fisher Scientific.

Preparation of sGP49 Surface Functioned AuNP Solution

The streptavidin surface functioned gold nanoparticles (0.13 nM, 80 μL) were first mixed with excessive amount of biotinylated sGP49 nanobody (1.2 μM, 25 μL). 2 hours of incubation ensured complete streptavidin-biotin conjugation. The mixture was then purified by centrifuge (accuSpin Micro 17, Thermo Fisher) at 10,000 rpm for 10 mins and repeated twice to remove unbounded biotinylated sGP49 nanobody. The purified AuNP solution was measured by Nanodrop 2000 (Thermo Fisher) to determine the final concentration. The concentration of AuNP solution was subsequently adjusted to 0.048 nM and the was aliquoted into 12 uL in a 500 uL Eppendorf tube. High concentration sGP solution (6 μM, in 1×PBS) underwent 10-fold serial dilution and 4 uL analyte solution of each concentration (4 pM to 4 uM) was mixed with 12 uL AuNP assay and vortexed (mini vortexer, Thermo Fisher) at 800 rpm for 5 seconds. The buffer used in assay preparation and sGP dilution was prepared by diluting 10×PBS buffer and mixing with glycerol and BSA to achieve final concentration of 1×PBS, 20% v/v glycerol and 1 wt % BSA buffer.

Centrifuge Enhanced Fast Detection Method

Same procedures were followed in preparation of sGP49 surface functioned AuNP solution. After mixing sGP49 surface functioned AuNP solution with sGP analyte solution, AuNP assay solution was centrifuged (accuSpin Micro 17, Thermo Fisher) at 3,500 rpm (1,200×g) for 1 minute. AuNPs were highly concentrated the bottom of Eppendorf tube. After 20 minutes of AuNP crosslinking, the assay solution was vortexed (mini vortexer, Thermo Fisher) at 800 rpm for 15 seconds to thoroughly resuspend un-crosslinked AuNP into solution.

PDMS Template Fabrication

Sylgard 184 silicone elastomer base (consisting of dimethyl vinyl-terminated dimethyl siloxane, dimethyl vinylated and trimethylated silica) was thoroughly mixed with curing agent (mass ratio 10:1) for 30 minutes and placed in a vacuum container for 2 hours to remove the bubbles generated during mixture. The mixture was then poured into a flat plastic container in room temperature and waited for one week until the PDMS is fully cured. The PDMS membrane was then cut to rectangle shape and holes were drilled by PDMS puncher to form sample cuvette. 2 mm diameter holes were drilled for Olympus BX53 microscope characterization using 50× objective lens. 4 mm diameter holes were drilled for Oceanview spectrometer characterization and Olympus BX53 microscope using 10× objective lens. To prevent non-specific bonding of proteins on PDMS membrane surface, the PDMS surface was pre-treated with PVA. The as prepared PDMS membrane and a rectangle shaped fused silica was first rinsed with isopropyl alcohol and oxygen plasma treated at a flow rate of 2 sccm and power of 75 W for 5 min. Immediately after oxygen plasma, the PDMS membrane was bonded to fused silica to form a PDMS template that can load assay solutions into the holes. Then the PDMS template was further oxygen plasma treated at a flow rate of 2 sccm and power of 75 W for 5 min and immediately soaked in 1% wt. PVA in water solution for 10 mins. The PDMS template was subsequently nitrogen blow dried and heated on 110° C. hotplate for 15 minutes. Finally, the PDMS template was removed from hotplate, nitrogen blow cooled and ready to use in UV-visible spectra characterization.

UV-Visible Spectra, SEM Imaging and Dark Field Scattering Imaging Characterizations

The UV-visible spectra were measured from a customized optical system. An Olympus BX53 microscope equipped with a LED light source (True Color, Olympus) was used to collect light signal from sample area. The PDMS template loaded solution samples were placed on the microscope sample stage and light transmitted through AuNP assay was collected by a 50× objective lens (NA=0.8). For drop cast samples on a glass slide, a 10× objective lens (NA=0.3) was used. The spectra were measured by a microscope-coupled UV-Vis-NIR spectrometer (iHR 320, Horiba) equipped with a CCD detector. The signal was scanned from 350 nm to 800 nm with integration time of 0.01 s and averaged for 64 times.

The SEM image was taken by Hitachi S4700 field emission scanning electron microscope at 5 keV and magnification of 15,000. For each drop cast sample with different concentrations, ten images from different area were taken. The size for the area taken in each image is 8.446 μm×5.913 μm.

The dark field scattering image was taken by Olympus BX53 microscope using an EMCCD camera (iXon Ultra, Andor). A xeon lamp (PowerArc, Horiba) was used for illumination light source. The light reflected from drop cast samples were collected by a 100× dark field lens (NA=0.9). The integration time was set to 50 ms. For each drop cast sample with different concentrations, ten images from different areas were taken. The size for the area taken in each image is 62.5 μm×62.5 μm.

UV-Visible Spectrometer Characterization of sGP in Diluted Fetal Bovine Serum

Fetal bovine serum was pre-diluted five fold by 1×PBS buffer to minimize serum matrix effect. sGP solution (6 μM, in 1×PBS) was diluted in series and added to diluted fetal bovine serum to make sGP in serum solution with concentration from 4 pM to 4 uM. 30 uL 0.048 nM sGP49 surface functioned 80 nm AuNP solution was then mixed with 10 uL sGP in serum solution and thoroughly vortexed. The mixture was then centrifuged following the protocol described in centrifuge enhanced fast detection method. The AuNP crosslinking time was 20 minutes. Next, the assay solutions were loaded into 4 mm diameter holes on a 3 mm thick PDMS template and characterized by Horiba iHR 320 UV-visible spectrometer. A 10× objective lens (NA=0.3) in Olympus BX53 microscope was used to collect the signal. The signals were averagely scanned from 350 nm to 800 nm for 64 times with integration time of 0.01 s. Next, the assay solutions were characterized by a miniaturized portable UV-visible spectra measurement system. OSL2 fiber coupled illuminator (Thorlabs) was used as light source. The light passed through the 4 mm diameter holes loaded with assay solution and coupled to Flame UV-visible miniaturized spectrometer (Ocean optics) for absorbance spectra measurement. The signals were averagely scanned from 430 nm to 1100 nm for 6 times and integrated for 5 seconds for each scan.

In the next step, a LED-photodetector measurement system was devised and demonstrated for sGP sensing with reduced equipment cost. The exemplary system includes three components, namely, an LED light source, a photodetector, and a centrifuge tube holder. The centrifuge tube holder was 3D printed using ABSplus P430 thermoplastic. An 8.6 mm diameter recess was designed to snug fit a standard 0.5 mL Eppendorf tube. A 2.8 mm diameter channel was open inside the centrifuge tube holder and aligned to the LED, assay colloid and photodetector, forming an optical path for signal measurement. The LED (597-3311-407NF, Dialight) was powered by two Duracell optimum AA batteries and a 35Ω resistor was serially connected as a voltage divider to set LED operating point. The photodetector (SFH 2270R, Osram Opto Semiconductors) was reversely biased by three Duracell optimum AA batteries and serially connected to a 7 MΩ load resistor. The photocurrent that responds to intensity of light transmitted through the assay was converted to voltage through a load resistor and measured by a portable multimeter (AstroAI AM33D).

CryoTEM Sample Preparation and Imaging

To image AuNP precipitates, the supernatant was removed from the tube and 2-3 uL of AuNP sample solution containing AuNP precipitates were left in the tube. The tube was then vortexed thoroughly. 2 uL of samples were pipetted and coated on both sides of an oxygen plasma treated Cu grid (Electron Microscopy Sciences, C flat, hole size 1.2 μm, hole spacing 1.3 μm). The oxygen plasma treatment time was 30 seconds. The Cu grid was plunge frozen in ethane using Vitrobot plunge freezer (FEI). The blot time was set to 6 seconds. After plunging, the sample was soaked in liquid nitrogen for long-term storage. For CryoTEM imaging, FEI Tecnai F20 transmission electron microscope was used with accelerating voltage set to 200 kV. More than 20 high-resolution TEM images were taken for 1 μM, 1 nM sGP in PBS samples and reference sample. The size for the area taken in each image is 4.476 μm×4.476 μm.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, kits, reaction mixtures, devices, and/or systems or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of detecting Ebola virus in a sample, the method comprising:
contacting the sample with a plurality of gold nanoparticles (AuNPs) and/or other plasmonic metal nanoparticles (MNPs) that are conjugated with one or more antibodies, or antigen binding portions thereof, that bind to an epitope of a secreted glycoprotein (sGP) from the Ebola virus under conditions sufficient for the one or more antibodies, or the antigen binding portions thereof, to bind to the epitope of the sGP from the Ebola virus in the sample to produce bound sGP; and,
detecting the sGP from the Ebola virus when one or more aggregations of the bound sGP form with one another, thereby detecting the Ebola virus in the sample.

2. (canceled)

3. (canceled)

4. The method of claim 1, wherein a given antibody, or antigen binding portion thereof, comprises an equilibrium dissociation constant (K_{D}) of between about 1 nM and about 100 nM.

5. The method of claim 1, wherein the plurality of AuNPs and/or other MNPs is conjugated with one or more nanobodies.

6. The method of claim 1, wherein the detection step comprises determining a change in absorbance at a resonance wavelength of the AuNPs and/or the other MNPs.

7. The method of claim 1, comprising quantifying an amount of the sGP and/or the Ebola virus in the sample.

8. The method of claim 1, further comprising centrifuging the aggregations of the bound sGP prior to and/or during the detecting step.

9. The method of claim 1, further comprising freezing the aggregations of the bound sGP prior to the detecting step.

10. The method of claim 1, comprising drop casting the aggregations of the bound sGP prior to the detecting step.

11. The method of claim 1, comprising obtaining the sample from a subject.

12. The method of claim 1, comprising administering one or more therapies to the subject when the Ebola virus is detected in the sample.

13. The method of claim 1, comprising detecting the Ebola virus within about 20 minutes or less of obtaining the sample from the subject.

14. The method of claim 1, comprising repeating the method using one or more longitudinal samples obtained from the subject.

15.-17. (canceled)

18. The method of claim 1, wherein the detecting step comprises measuring a colorimetric change when the one or more aggregations of the bound sGP form with one another.

19. The method of claim 1, comprising visually detecting the colorimetric change when the one or more aggregations of the bound sGP form with one another.

20. The method of claim 1, comprising detecting the colorimetric change when the one or more aggregations of the bound sGP form with one another using a spectrometer.

21. The method of claim 1, wherein a concentration of sGP in the sample is from about 1 μM to about 100 fM.

22.-27. (canceled)

28. A system, comprising:
a device, comprising at least one reaction chamber or substrate comprising a plurality of gold nanoparticles (AuNPs) and/or other plasmonic metal nanoparticles (MNPs) that are conjugated with one or more antibodies, or antigen binding portions thereof, that bind to an epitope of a secreted glycoprotein (sGP) from an Ebola virus when the reaction chamber receives a sample that comprises the Ebola virus under conditions sufficient for the one or more antibodies, or the antigen binding portions thereof, to bind to the epitope of the sGP from the Ebola virus in the sample to produce bound sGP; and,
an electromagnetic radiation detection apparatus positioned, or positionable, within sufficient proximity to the device to detect one or more colorimetric changes produced in or on the reaction chamber or substrate when one or more aggregations of the bound sGP form with one another in or on the reaction chamber or substrate.

29. The system of claim 28, wherein the electromagnetic radiation detection apparatus comprises a spectrometer, a microscope, or a light-emitting diode (LED) that transmits light into and/or through the reaction chamber or substrate and a photodetector that detects light from the reaction chamber or substrate.

30.-32. (canceled)

33. A method of detecting small molecules or cells in a sample, the method comprising:
contacting the sample with a plurality of gold nanoparticles (AuNPs) and/or other plasmonic metal nanoparticles (MNPs) that are conjugated with one or more antibodies, or antigen binding portions thereof, that bind to an epitope of a small molecule or a cell under conditions sufficient for the one or more antibodies, or the antigen binding portions thereof, to bind to the epitope of the small molecule or the cell in the sample to produce bound small molecules or cells; and,
detecting the small molecules or the cells when one or more aggregations of the bound small molecules or cells form with one another, thereby detecting the small molecules or the cells in the sample.

34. The method of claim 33, wherein a given antibody, or antigen binding portion thereof, comprises an equilibrium dissociation constant (K_{D}) of between about 1 nM and about 100 nM.

35. The method of claim 33, wherein the plurality of AuNPs and/or other MNPs is conjugated with one or more nanobodies.

36. The method of claim 33, wherein the detection step comprises determining a change in absorbance at a resonance wavelength of the AuNPs and/or the other MNPs.

37. The method of claim 33, comprising quantifying an amount of the small molecules or the cells in the sample.

38. The method of claim 33, further comprising centrifuging the aggregations of the bound small molecules or cells prior to and/or during the detecting step.

39. The method of claim 33, further comprising freezing the aggregations of the bound small molecules or cells prior to the detecting step.

40. The method of claim 33, comprising drop casting the aggregations of the bound small molecules or cells prior to the detecting step.

41. The method of claim 33, comprising obtaining the sample from a subject.

42. The method of claim 33, comprising administering one or more therapies to the subject when the small molecules or cells are detected in the sample.

43. The method of claim 33, comprising detecting the small molecules or the cells within about 20 minutes or less of obtaining the sample from the subject.

44. The method of claim 33, comprising repeating the method using one or more longitudinal samples obtained from the subject.

45. The method of claim 33, wherein the detecting step comprises measuring a colorimetric change when the one or more aggregations of the bound small molecules or cells form with one another.

46. The method of claim 33, comprising visually detecting the colorimetric change when the one or more aggregations of the bound small molecules or cells form with one another.

47. The method of claim 33, comprising detecting the colorimetric change when the one or more aggregations of the bound small molecules or cells form with one another using a spectrometer.

48. The method of claim 33, wherein a concentration of the small molecules or the cells in the sample is from about 1 μM to about 100 fM.

49. A system, comprising:
a device, comprising at least one reaction chamber or substrate comprising a plurality of gold nanoparticles (AuNPs) and/or other plasmonic metal nanoparticles (MNPs) that are conjugated with one or more antibodies, or antigen binding portions thereof, that bind to an epitope of a small molecule or a cell when the reaction chamber receives a sample that comprises the small molecule or the cell under conditions sufficient for the one or more antibodies, or the antigen binding portions thereof, to bind to the epitope of the small molecule or the cell in the sample to produce bound small molecules or cells; and,
an electromagnetic radiation detection apparatus positioned, or positionable, within sufficient proximity to the device to detect one or more colorimetric changes produced in or on the reaction chamber or substrate when one or more aggregations of the bound small molecules or cells form with one another in or on the reaction chamber or substrate.

50. The system of claim 49, wherein the electromagnetic radiation detection apparatus comprises a spectrometer, a microscope, or a light-emitting diode (LED) that transmits light into and/or through the reaction chamber or substrate and a photodetector that detects light from the reaction chamber or substrate.
